(19) United States
(12) Patent Application Publication
(10) Pub. No.: US 2008/0234717 A1
(43) Pub. Date: Sep. 25, 2008

(54) Helical Screw Puncture Tip

(75) Inventor: Walter Bruszewski, Guerneville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/688,503

(22) Filed: Mar. 20, 2007

(51) Int. Cl. A61M 29/00; A61B 17/56; A61B 17/04; A61B 17/32

(52) U.S. Cl. 606/191; 606/304; 606/321; 606/167

(57) ABSTRACT

A helical screw dilator system and method including a helical screw dilator having a proximal end, a distal end, and an axial length, the helical screw dilator including a piercing tip at the distal end; a threaded dilation portion operably connected to the piercing tip along the axial length, the threaded dilation portion having an outer surface increasing in circumference with distance from the distal end and having threads disposed about the outer surface; and a shaft between the threaded dilation portion and the proximal end and a guidewire lumen through the center to guide the system to its destination.

### TECHNICAL FIELD

The technical field of this disclosure is medical implantation devices, particularly, a helical screw dilator system and method.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include arteries such as those located within coronary, mesentery, peripheral, or cerebral vasculature; veins; gastrointestinal tract; biliary tract; urethra; trachea; hepatic shunts; and fallopian tubes. Various types of endoluminal prostheses have also been developed to modify the mechanics of the targeted luminal wall.

A number of vascular devices have been developed for replacing, supplementing, or excluding portions of blood vessels. These vascular devices include endoluminal vascular prostheses and stent grafts. Aneurysm exclusion devices, such as abdominal aortic aneurysm (AAA) devices, are used to exclude vascular aneurysms and provide a prosthetic lumen for the flow of blood. Vascular aneurysms are the result of abnormal dilation of a blood vessel, usually from disease or a genetic predisposition, which can weaken the arterial wall and allow it to expand. Aneurysms can occur in any blood vessel, but most occur in the aorta and peripheral arteries, with the majority of aortic aneurysms occurring in the abdominal aorta. An abdominal aortic aneurysm typically begins below the renal arteries and extends into one or both of the iliac arteries.

Aneurysms, especially abdominal aortic aneurysms, are commonly treated in open surgery procedures in which the diseased vessel segment is bypassed and repaired with an artificial vascular graft. While open surgery is an effective surgical technique in light of the risk of a fatal abdominal aortic aneurysm rupture, the open surgical technique suffers from a number of disadvantages. The surgical procedure is complex, requires a long hospital stay, requires a long recovery time, and has a high mortality rate. Less invasive devices and techniques have been developed to avoid these disadvantages. Tubular endoluminal prostheses that provide a lumen or lumens for blood flow while excluding blood flow to the aneurysm site are introduced into the blood vessel using a catheter in a minimally invasive technique. The tubular endoluminal prosthesis is introduced in a small diameter compressed condition and expanded at the aneurysm. Although often referred to as stent grafts, these tubular endoluminal prostheses differ from covered stents in that they are not used to mechanically prop open natural blood vessels. Rather, they are used to secure an artificial lumen in a sealing engagement with the vessel wall without further opening the abnormally dilated natural blood vessel.

Stent grafts typically include a support framework supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer surfaces of the support structure (framework), which supports the graft material and/or holds it in place against a luminal wall. The stent graft is secured to a vessel wall above and below the aneurysm. A proximal spring stent of the stent graft can be located above the aneurysm to provide a radial force which engages the lumen wall and seals the stent graft at the lumen wall. The proximal spring stent can include hooks to puncture the vessel wall and further secure the stent graft in place.

One problem in present stent graft designs is the need to fix the proximal spring stent superior to the renal arteries and superior mesenteric artery when the only region suitable for sealing is superior to these visceral arteries. An estimated ten percent of AAA cases amenable to endovascular repair require suprarenal fixation, cutting off blood to the kidneys and intestine. One proposed solution to this problem has been to provide branched conduits from the stent graft in the aorta to perfuse the renal arteries and superior mesenteric artery.

Unfortunately, the anatomy of the branching of the renal arteries and superior mesenteric artery varies from patient to patient. The axial location, axial angle, and radial angle of the branch vessels all can vary. One approach to this problem has been to custom make a stent graft with branched conduits for a particular patient. This approach increases complexity since each stent graft is different, requiring personal measurement and tailoring. In addition, the customized stent grafts are difficult to deploy, since the branch conduits are attached before deployment and thereby increase the packing density in the delivery catheter or require use of a delivery catheter with a larger diameter than might be used with the main device alone. The efficacy of customized stent grafts is yet to be proven.

Another approach to the problem of variable anatomy has been to fenestrate the graft material in situ after the stent has been deployed, forming a fenestration to provide a passage between the stent graft lumen and the visceral arteries. The general approach has been to pierce the graft material at the location of the branch vessel to be perfused and to work the hole until it is the size desired. In one case, a needle is used to pierce the graft material and a larger needle used to dilate the needle hole. A balloon is then used to enlarge the dilated hole to a final diameter. A covered stent can be deployed in the hole to provide a flow path between the stent graft lumen and the visceral artery, and to maintain patency of the visceral artery.

One difficulty with in situ fenestration is the amount of force required to dilate the needle hole. The graft material is tough so that excessive axial force is required to dilate the needle hole. This reduces the control of the attending physician and can even result in inadvertent puncture of the vessel wall with the dilator if a slip should occur. The situation is exacerbated by the geometry of the aortic artery and the branch artery. The catheter delivering the dilator runs along the axis of the aortic artery, and then turns at 90 degrees or more so that the point of the dilator is perpendicular to the wall of the stent graft. The turn affects the force that can be applied on the dilator, so that it may not be possible to apply sufficient force to the dilator to increase the diameter of the needle hole to the size desired.

A difficulty with conventional conical dilators is that the graft cloth (material) resists passage of the dilator. Thus the considerable force required to urge the dilator through the graft cloth is translated into deformation of the cloth. This can disturb the position of the main stent graft.

It would be desirable to have a dilator system and method that would overcome the above disadvantages.

### SUMMARY OF THE INVENTION

One aspect according to the present invention provides a helical screw dilator having a proximal end, a distal end, and an axial length, the helical screw dilator including a piercing tip at the distal end; a threaded dilation portion operably connected to the piercing tip along the axial length, the threaded dilation portion having an outer surface increasing in circumference with distance from the distal end and having threads disposed about the outer surface; and a shaft between the threaded dilation portion and the proximal end.

Another aspect provides a helical screw dilator system for use in a vasculature of a patient, the helical screw dilator system including a helical screw dilator having a proximal end, a distal end, an axial length, and an axial lumen along the axial length, the helical screw dilator comprising a piercing tip at the distal end; a threaded dilation portion connected to the piercing tip, the threaded dilation portion having an outer surface increasing in circumference with distance from the distal end and having threads disposed about the outer surface; and a shaft between the threaded dilation portion and the proximal end; a steerable catheter adapted to receive and guide the helical screw dilator in the vasculature; and a guidewire insertable in the axial lumen.

Another aspect provides a method of stent graft fenestration in a vasculature, the method including providing a stent graft having graft material; deploying the stent graft in the vasculature; providing a helical screw dilator having a proximal end, a distal end, and an axial length, the helical screw dilator comprising a piercing tip at the distal end; a threaded dilation portion connected to the piercing tip, the threaded dilation portion having an outer surface increasing in circumference with distance from the distal end and having threads disposed about the outer surface; and a shaft between the threaded dilation portion and the proximal end; forming a hole in the graft material with the piercing tip; engaging the threads with the hole; and rotating the threaded dilation portion to advance the threaded dilation portion into the hole.

Another aspect provides helical screw dilator for graft material of a stent graft including means for piercing a hole in the graft material; means for dilating the hole; means for advancing the dilating means into the hole in response to rotation; and means for rotating the advancing means.

The foregoing and other features and advantages will become further apparent from the following detailed description, read in conjunction with the accompanying drawings.

### BRIEF DESCRIPTION OF THE DRAWINGS

FIG. 1 is a side view of a helical screw dilator system in accordance with the present invention;

FIG. 2 is a close-up view of the distal end of a helical screw dilator in accordance with the present invention;

FIG. 3 is a cross section view of a helical screw dilator in accordance with the present invention;

FIG. 4 is a cross section view of another embodiment of a helical screw dilator made in accordance with the present invention;

FIG. 5 is a schematicized close-up view of a helical screw dilator dilating a hole;

FIG. 6 is a view of a hole made by a helical screw dilator;

FIG. 7 is a block diagram of an electrical circuit for hole sealing for a helical screw dilator in accordance with the present invention; and

FIG. 8 is a flowchart of a method of using a helical screw dilator.

### DETAILED DESCRIPTION

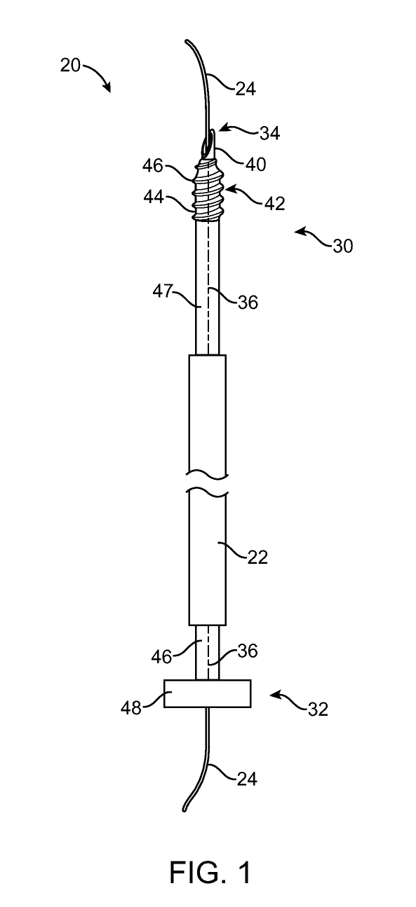

FIG. 1 is a side view of a helical screw dilator system in accordance with the present invention. The helical screw dilator system 20 includes a helical screw dilator 30, a steerable catheter 22, and a guidewire 24. The helical screw dilator 30 can be used to make a hole in material, such as graft material in a stent graft. Those skilled in the art will appreciate that the helical screw dilator 30 can also be used to make holes in tissue or other materials where precise control is required and axial force needs to be limited. For example, the helical screw dilator 30 can be used to make a hole in a vessel or between heart chambers.

The helical screw dilator system 20 has a proximal end 32, a distal end 34, and an axial length 36, along the central axis of the helical screw dilator 30 and indicated by the dashed line between the proximal end 32 and the distal end 34. As used herein, the proximal end 32 is defined as the end of the helical screw dilator 30 nearer the operator when the helical screw dilator 30 is in place in the vasculature of a patient and the distal end 34 is defined as the end of the helical screw dilator 30 at the fenestration site. The helical screw dilator 30 includes a piercing tip 40 at the distal end 34; a threaded dilation portion 42 connected to the piercing tip 40 along the axial length 36, the threaded dilation portion 42 having an outer surface 44 increasing in circumference with distance from the distal end 34 and having threads 46 disposed about the outer surface 44; and a shaft 47 between the threaded dilation portion 42 and the proximal end 32. In one embodiment, the helical screw dilator system 20 includes a handle 48 at the proximal end 32 to allow the operator to apply axial force and/or torque to the helical screw dilator 30 through the shaft 47.

The helical screw dilator 30 can be used for in situ fenestration of an already deployed stent graft whose body structure covers one or more openings (ostia) of a branch vessel from the main aortic vessel. The steerable catheter 22 is advanced to the puncture site within the stent graft already deployed in the abdominal aortic aneurysm, through the femoral artery, the carotid artery, or the subclavian artery. The tip of the steerable catheter 22 can be steerable in one or two directions. The steerable catheter 22 is guided to the intended deployment location of the stent graft with X-ray or fluoroscopic data. The helical screw dilator 30 is advanced through the steerable catheter 22 to the fenestration site within the lumen of the stent graft. The fenestration site can be at the ostium of a renal artery in which a branch conduit is to be installed to maintain perfusion. The steerable catheter 22 directs the helical screw dilator 30 to the fenestration site under the control of the operator. In response to axial force along the shaft 47, the piercing tip 40 passes through the fenestration site in the graft material to form a hole. The threaded dilation portion 42 of the helical screw dilator 30 is advanced to engage the threads 46 with the hole. The threaded dilation portion 42 is rotated in the hole to advance the threaded dilation portion 42 into the hole and enlarge the hole about the outer surface 44 of the threaded dilation portion 42 (like a self tapping screw). The rotation is stopped when the hole is the desired size. The shaft 47 is rotated slightly in a reverse direction to back the threads out of the hole and the helical screw dilator 30 is removed from the steerable catheter 22. The helical screw dilator 30 can be removed, and an angioplasty balloon can then be inserted in the dilated hole via a steerable catheter 22. Finally, a branch conduit installed in the fenestration, and the steerable catheter 22 removed from the patient.

In one embodiment, the axial length 36 of the helical screw dilator 30 includes an axial lumen to receive the guidewire 24, which assists in alignment of the threaded dilation portion 42 of the helical screw dilator 30 with the fenestration site or the hole formed by the piercing tip 40 in the graft material. When the piercing tip 40 is retractable, the piercing tip 40 can be retracted into the threaded dilation portion 42 once the hole is made. The guidewire 24 is advanced into the hole through the piercing tip 40 before the piercing tip 40 is retracted and keeps the hole open and maintains alignment so the threads 46 can engage the hole after the piercing tip 40 is retracted.

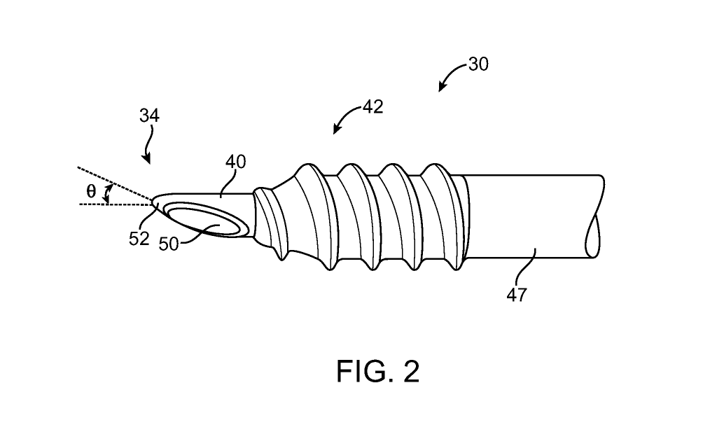

FIG. 2 in which like elements share like reference numbers with FIG. 1, is a close-up view of the distal end of a helical screw dilator in accordance with the present invention. The helical screw dilator 30 includes a piercing tip 40, a threaded dilation portion 42 with an outer surface 44 and threads 46 disposed about the outer surface 44, and a shaft 47. The outer surface 44 of the threaded dilation portion 42 increases in circumference with distance from the distal end 34, so that the threaded dilation portion 42 can increase the diameter of a hole pierced by the piercing tip 40 in graft material or tissue as the threaded dilation portion 42 is screwed into the hole.

The piercing tip 40 is used to form a hole in the graft material or tissue at the fenestration site. In one embodiment, the piercing tip 40 is fixed and remains in position beyond the threaded dilation portion 42 after the piercing tip 40 forms the hole. In another embodiment, the piercing tip 40 is retractable and can be withdrawn into the threaded dilation portion 42 after the piercing tip 40 forms the hole. The piercing tip 40 can be retracted by a mechanism extending to the proximal end of the helical screw dilator 30, such as a shaft or cable. A retractable piercing tip 40 assures that the piercing tip 40 will not inadvertently pierce or damage tissue beyond the hole as the threaded dilation portion 42 is rotated and advances into the hole.

The piercing tip 40 can be any configuration suitable for piercing the particular graft material or tissue with which it is to be used. The piercing tip 40 forms the initial hole in the graft material or tissue at the fenestration site. The piercing tip 40 can optionally include an tip lumen 50 for passage of a guidewire and can be fabricated from a needle, a hypotube, or the like. The piercing tip 40 can be beveled at an angle E between the axial length and the bevel face to provide a pointed tip 52 on the piercing tip 40. In one example, the piercing tip 40 is formed from a stainless steel biopsy needle with a diameter of 0.053 inches and the bevel angle θ is 18 degrees. In another embodiment, the piercing tip 40 is solid and has no lumen. In yet another embodiment, the piercing tip 40 can include patterns or cutting surfaces suitable for the graft material or tissue to be pierced. In the embodiment in which the piercing tip is fixed to the threaded dilation portion, the piercing tip 40 can be attached to the threaded dilation portion 42 by welding, laser welding, brazing, adhesive fixing, mechanical interference or by any other suitable method of attaching the parts. The piercing tip 40 can be made of spring steel, stainless steel, titanium, nitinol, polymers or copolymers, combinations thereof, or other suitable materials.

The threaded dilation portion 42 has an outer surface 44 increasing in circumference with distance from the distal end 34 and has threads 46 disposed about the outer surface 44. The threaded dilation portion 42 dilates the initial hole formed in the graft material or tissue at the fenestration site. The threads 46 (which may have a sharp edge to make cutting the graft material easier) engage the graft material or tissue and the outer surface 44 enlarges the hole as the threaded dilation portion 42 is rotated by the shaft 47 and advances into the hole. The threaded dilation portion 42 can optionally include a dilation portion lumen (not shown) communicating with the tip lumen 50 for passage of a guidewire. In another embodiment, the threaded dilation portion 42 is solid and without a lumen.

The characteristics of the threads 46 and taper of the outer surface 44 can be selected to suit the graft material or tissue to be fenestrated. The threads 46 can be coarse with a low pitch, such as the thread of a No. 8 sheet metal screw. The threads can be a Type A or AB thread-forming (sheet metal) screw as described in ANSI Standard B18.6.4-1981, R1991. Amongst such screw designs, a coarser, deeper thread is likely to be more effective in the function of displacing graft cloth with increasing minor diameter as the threads engage the graft cloth or tissue. A pitch approaching the type A standard (for example, eighteen or fewer threads per inch for a size 6 screw) is likely to function more effectively at engaging tissue or graft cloth. As well, thread depth approaching the type A standard (ratio of major diameter to minor diameter of 1.38 or greater for a size 6 screw, for example), is expected to provide good engagement. An exemplary screw thread form is Acme or Unified with a flat root. The angle between flanks for both type A and AB is 60 degrees. An embodiment of the dilator has a gimlet tip design. This is also specified in the above standard for type A and AB sheet metal screws. This tip design is characterized as conical (with a 45 degree included angle) with threads extending to the end of the tip. It is likely that progressively coarser pitch of thread approaching the conical tip would result in more effective initial engagement of the dilator. The threads 46 pull the threaded dilation portion 42 into the graft material or tissue. A low pitch with a gentle taper allows the threaded dilation portion 42 to start easily in the initial hole formed by the piercing tip 40. The taper of the outer surface 44 gradually increases the diameter of the hole at the fenestration site. The taper increases until the diameter of the outer surface 44 is the diameter desired for the final hole. In one example, 0.5 inch-pounds torque is required to dilate a 0.05 inch diameter initial hole to a 0.115 inch diameter final hole, which is large enough to deploy an 8 Fr. diameter coated stent over a guidewire. Those skilled in the art will appreciate that the thread and taper can be selected as desired for a particular application.

The threaded dilation portion 42 can be attached to the shaft 47 by welding, laser welding, brazing, adhesive fixing, or by any other suitable method of attaching the parts. The threaded dilation portion 42 can be made of spring steel, stainless steel, titanium, nitinol, platinum, platinum iridium alloy, polymers or copolymers, combinations thereof, or other suitable materials. In one example, the threaded dilation portion 42 is made of stainless steel.

The shaft 47 can be any flexible shaft capable of rotating the threaded dilation portion 42 and capable of transmitting an axial force to push the piercing tip 40 through the graft material or tissue. The shaft 47 is bendable in all directions. In one example, the shaft 47 is a stainless steel, laser slotted hypotube. The shaft 47 can be made of spring steel, stainless steel, titanium, nitinol, a polymer or copolymer, a combination of these materials, or other suitable materials. In another example the shaft is a solid or hollow torque shaft consisting of helically wound wires or a braided shaft. The shaft 47 can optionally include a shaft lumen (not shown) communicating with the dilation portion lumen for passage of a guidewire. In another embodiment, the shaft 47 is solid and without a lumen. The shaft 47 can be driven manually with a handle or with a power drive.

The helical screw dilator 30 can include radiopaque markers as desired to improve visibility on X-ray and fluoroscopic images. The radiopaque markers can be made of platinum, iridium, or any other radiopaque material. In one embodiment, a radiopaque marker (a ring, a pin, or other suitable geometric arrangement) is provided where the piercing tip 40 joins the threaded dilation portion 42. In another embodiment, a radiopaque marker is provided where the threaded dilation portion 42 joins the shaft 47. Those skilled in the art will appreciate that the radiopaque markers may not be required when the helical screw dilator 30 includes radiopaque materials.

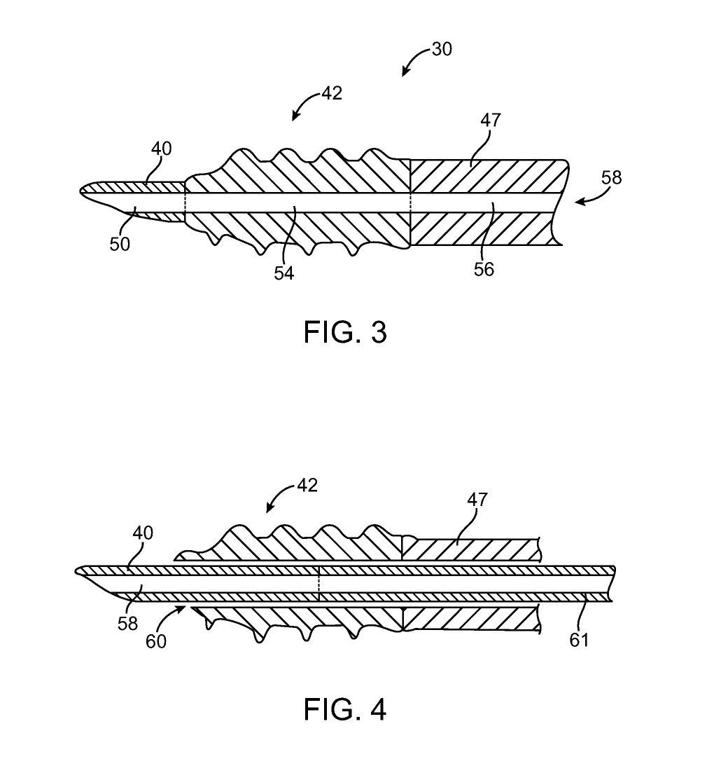

FIG. 3, in which like elements share like reference numbers with FIG. 2, is a cross section view of a helical screw dilator. In this embodiment, the helical screw dilator 30 has a fixed piercing tip 40 and an axial lumen 58 for passage of a guidewire. The axial lumen 58 includes a tip lumen 50, a dilation portion lumen 54, and a shaft lumen 56.

FIG. 4, in which like elements share like reference numbers with FIG. 2, is a cross section view of another embodiment of a helical screw dilator. In this embodiment, the helical screw dilator 30 has a retractable piercing tip 40 and an axial lumen 58 for passage of a guidewire. The piercing tip 40 is slidably disposed in a retractable tip lumen 60 that runs the length of the helical screw dilator 30. A flexible tip shaft 61 is connected to the piercing tip 40 to allow operation of the retractable piercing tip 40. The operator can slide the flexible tip shaft 61 to expose the piercing tip 40 for forming the hole in the graft material or tissue at the fenestration site and for retracting the piercing tip 40 into the threaded dilation portion 42 after the initial hole has been made. With the piercing tip 40 retracted, the threaded dilation portion 42 can be used to dilate the hole without concern that the piercing tip 40 could inadvertently pierce or cut other tissue beyond the fenestration site.

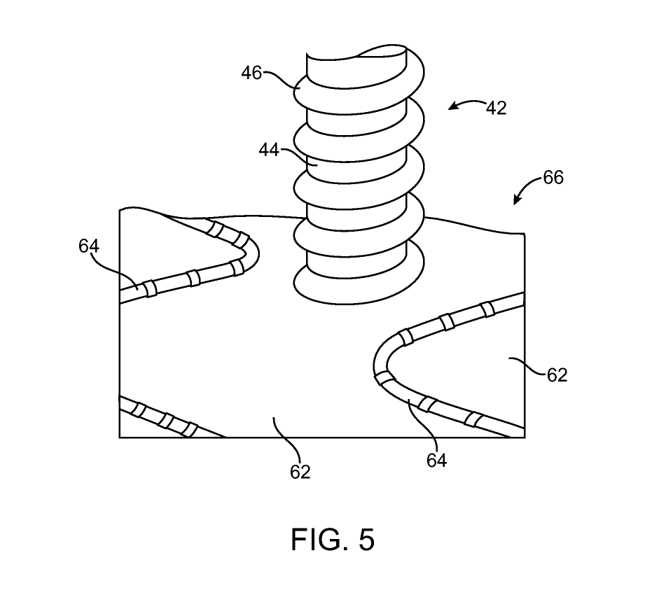

FIG. 5, in which like elements share like reference numbers with FIG. 2, is a detailed view of a helical screw dilator dilating a hole. The threaded dilation portion 42 is dilating the initial hole in graft material 62 of a stent graft 66. The stent graft 66 includes the graft material 62 and support (framework) structure 64. In this example, the graft material 62 is a high density woven Dacron® polyester. In one embodiment, the threaded dilation portion 42 can be heated by inductance or resistance while the threaded dilation portion 42 is maintained in the final-sized hole to cause localized melting of the frayed end of the material to seal the edges of the hole in the graft material 62. Those skilled in the art will appreciate that the graft material can be any woven or interlocked graft material suitable for stent grafts, such as woven polymer materials, e.g., Dacron, or polytetrafluoroethylene (PTFE), or interlocked graft materials including knit, braided stretch, and velour materials.

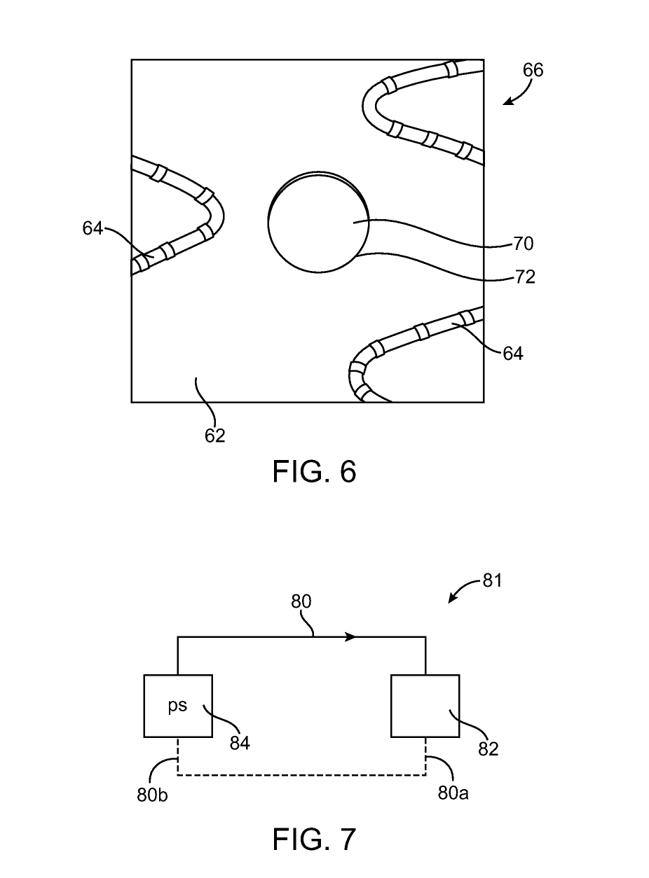

FIG. 6, in which like elements share like reference numbers with FIG. 5, is a detailed view a hole made by a helical screw dilator. The hole 70 has a regular circumference 72 and exhibits little fraying. Those skilled in the art will appreciate that the helical screw dilator can be used for procedures other than fenestration of stent grafts. The helical screw dilator can be used to form holes in tissue as well as graft material. In one example, the helical screw dilator can be used to form a hole in a vessel for anastomosis, i.e., the joining of two vessels. In another example, the helical screw dilator can be used to make a path for transeptal cardiac procedures. In an additional example the helical screw dilator can be used to create an opening in the skin, subcutaneous tissue, muscle, and serosa to accommodate a laparoscopic port for minimally invasive catheter access.

FIG. 7 is a block diagram of an electrical circuit for hole sealing for a helical screw dilator. The electrical circuit 81 provides a current 80 through the threaded dilation portion 82 of the helical screw dilator from a power source 84. The threaded dilation portion 82 can be electrically connected across the power source 84 by a conductor running through axial length of the helical screw dilator. The current 80 heats the threaded dilation portion 82, which remains in the hole after the hole has been dilated to a final diameter. The return leg of the circuit 80a is completed through the conductive medium of the blood or tissue fluid to a common electrode 80b. The heated threaded dilation portion 82 seals the edge of the hole by softening or melting the graft material at the edge. In an alternate embodiment, the threaded dilation portion 82 is heated inductively with a radiofrequency wave directed toward the threaded dilation portion 82 from outside the patient. Those skilled in the art will appreciate that the time and temperature for heating of the threaded dilation portion can be selected as desired for the particular application and materials involved.

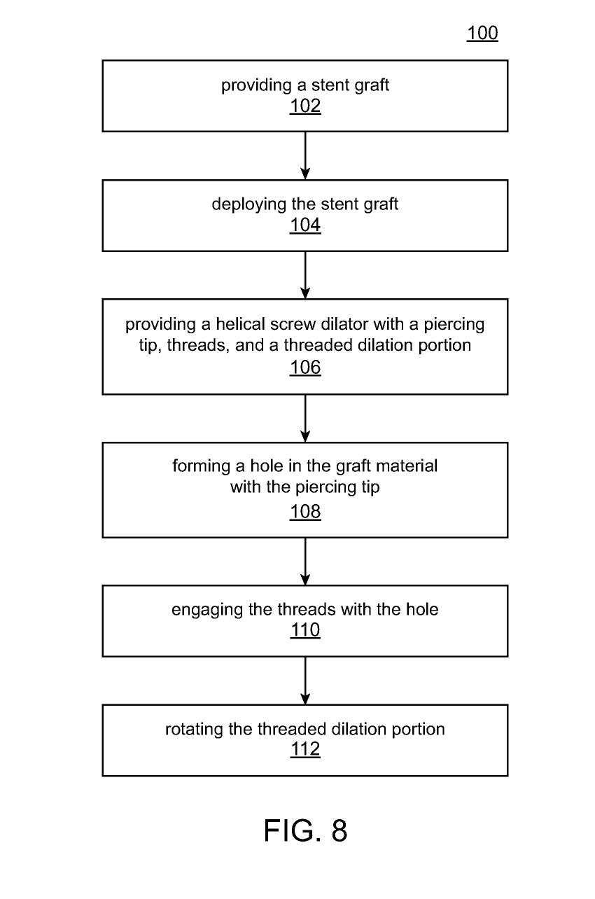

FIG. 8 is a flowchart setting out steps of a method of using a helical screw dilator made in accordance with the present invention. The method 100 may include providing a stent graft 102 and may include; deploying the stent graft 104; providing a helical screw dilator with a piercing tip, threads, and a threaded dilation portion 106; forming a hole in the graft material with the piercing tip 108, engaging the threads with the hole 110; and rotating the threaded dilation portion 112.

The prerequisite step/condition of providing a stent graft 102 includes providing a stent graft having graft material. The graft material can provide a pressure boundary in the vasculature of a patient, such as in an aortic aneurysm. Deploying the stent graft 104 includes deploying the stent graft in the vasculature.

The step of providing a helical screw dilator with a piercing tip, threads, and a threaded dilation portion 106 includes providing a helical screw dilator having a proximal end, a distal end, and an axial length, the helical screw dilator including a piercing tip at the distal end; a threaded dilation portion connected to the piercing tip, the threaded dilation portion having an outer surface increasing in circumference with distance from the distal end and having threads disposed about the outer surface; and a shaft between the threaded dilation portion and the proximal end. Exemplary helical screw dilators are illustrated in FIGS. 1-4 above.

Referring to FIG. 8, forming a hole in the graft material with the piercing tip 108 includes the step of forming a hole in the graft material with the piercing tip at a fenestration site. The piercing tip of the helical screw dilator can be advanced along the axial length of the helical screw dilator to advance the piercing tip and form the hole. In one embodiment, the helical screw dilator is advanced to the fenestration site through a steerable catheter that directs the piercing tip to the fenestration site at the graft material. The fenestration site can be the location of a branching vessel, such as a renal artery off the abdominal aorta. In one embodiment, the step of forming a hole can also include rotating the piercing tip to help form the hole. When the piercing tip is retractable, the piercing tip can be retracted after forming the hole. When the helical screw dilator includes an axial lumen, the piercing tip can be directed to the fenestration site over a steerable guide wire placed in the axial lumen. The steerable guide wire can also be placed in the hole formed by a retractable piercing tip to hold the hole open and direct the threaded dilation portion to the hole after the piercing tip is retracted.

The step of engaging the threads with the hole 110 includes advancing the threaded dilation portion of the helical screw dilator into the hole. The step of rotating the threaded dilation portion 112 includes rotating the threaded dilation portion to advance the threaded dilation portion into the hole. As the threaded dilation portion advances, its increasing circumference dilates the hole. In one embodiment, the threaded dilation portion is heated by inductance or resistance after the hole has reached the final diameter to seal the edges of the hole. The threaded dilation portion can be counter-rotated to free the threaded dilation portion from the final hole and the helical screw dilator removed from the patient.

The device may also be described as a helical screw dilator for graft material of a stent graft that includes: a piercer (means for) piercing a hole in the graft material; a dilator (means for) dilating the hole; an advancer (means for) advancing the dilator into the hole in response to rotation; a rotator (means for) rotating the advancer, a lumen (means for) receiving a guidewire through the piercer, the dilator, and the rotator, and a retractor (means for) retracting the piercer.

While specific embodiments according to the invention are disclosed herein, various changes and modifications can be made without departing from the spirit and scope of the invention.

1. A helical screw dilator having a proximal end, a distal end, and an axial length, the helical screw dilator comprising:
a threaded dilation portion having an outer surface increasing in circumference with distance from the distal end and having threads disposed about the outer surface; and
a shaft between the threaded dilation portion and the proximal end.

2. The helical screw dilator of claim 1 wherein the helical screw dilator has an axial lumen along the axial length therethrough.

3. The helical screw dilator of claim 2 further comprising a piercing tip at the distal end, wherein said threaded dilation portion is operably connected to the piercing tip along the axial length.

4. The helical screw dilator of claim 1 wherein the piercing tip is fixed to the threaded dilation portion.

5. The helical dilator of claim 4 wherein the helical screw dilator piercing tip is retractable into the threaded dilation portion, the forming further comprising:

6. The helical screw dilator of claim 4 wherein said axial lumen includes a tip lumen in the piercing tip and a dilation portion lumen in the threaded dilation portion, where the dilation portion lumen is in communication with the tip lumen.

7. The helical screw dilator of claim 4 wherein the piercing tip is fixed to the threaded dilation portion by an attachment selected from the group consisting of weld, laser weld, mechanical interference, brazed joint, and adhesive.

8. The helical screw dilator of claim 2 wherein the threaded dilation portion has a retractable tip lumen and the piercing tip is slidably disposed in the retractable tip lumen.

9. The helical screw dilator of claim 8 wherein the piercing tip has an axial lumen.

10. The helical screw dilator of claim 3 wherein the piercing tip is made of a material selected from the group consisting of spring steel, stainless steel, titanium, nitinol, polymers, copolymers, and combinations thereof.

11. The helical screw dilator of claim 3 wherein the threads are No. 8 sheet metal screw threads.

12. The helical screw dilator of claim 3 wherein the threaded dilation portion is made of a material selected from the group consisting of spring steel, stainless steel, titanium, nitinol, and combinations thereof.

13. The helical screw dilator of claim 3 wherein the shaft is a slotted hypotube.

14. The helical screw dilator of claim 3 wherein the shaft is a torque tube.

15. The helical screw dilator of claim 3 wherein the shaft is a braided tube.

16. The helical screw dilator of claim 3 further comprising a radiopaque marker disposed at a junction of the piercing tip and the threaded dilation portion.

17. The helical screw dilator of claim 1 further comprising a radiopaque marker disposed at a junction of the threaded dilation portion and the shaft.

18. The helical screw dilator of claim 3 further comprising a power source electrically connected across the threaded dilation portion.

19. A helical screw dilator system for use in a vasculature of a patient, the helical screw dilator system comprising:
a helical screw dilator having a proximal end, a distal end, an axial length, and an axial lumen along the axial length, the helical screw dilator comprising a piercing tip at the distal end; a threaded dilation portion connected to the piercing tip, the threaded dilation portion having an outer surface increasing in circumference with distance from the distal end and having threads disposed about the outer surface; and a shaft between the threaded dilation portion and the proximal end;
a steerable catheter adapted to receive and guide the helical screw dilator in the vasculature; and
a guidewire insertable in the axial lumen.

20. The helical screw dilator system of claim 19 wherein the piercing tip is fixed to the threaded dilation portion.

21. The helical screw dilator system of claim 19 wherein the threaded dilation portion has a retractable tip lumen and the piercing tip is slidably disposed in the retractable tip lumen.

22. The helical screw dilator system of claim 19 wherein the shaft is a slotted hypotube.

23. The helical screw dilator system of claim 19 further comprising a power source electrically connected across the threaded dilation portion.

24. A method of stent graft fenestration in a vasculature, the method comprising:
providing a helical screw dilator having a proximal end, a distal end, and an axial length, the helical screw dilator comprising a piercing tip at the distal end; a threaded dilation portion connected to the piercing tip, the threaded dilation portion having an outer surface increasing in circumference with distance from the distal end and having threads disposed about the outer surface; and a shaft between the threaded dilation portion and the proximal end;
forming a hole in a graft material with the piercing tip;
engaging the threads with the hole; and
rotating the threaded dilation portion to advance the threaded dilation portion into the hole.

25. The method of claim 24 wherein the helical screw dilator has an axial lumen along the axial length therethrough.

26. The method of claim 25 further comprising the steps of:
providing a stent graft having graft material;
deploying the stent graft in the vasculature;

27. The method of claim 25 wherein the forming comprises advancing the piercing tip along the axial length.

28. The method of claim 27 wherein the step of forming a hole further comprises rotating the piercing tip.

29. The method of claim 25 wherein the helical screw dilator piercing tip is retractable into the threaded dilation portion, the forming further comprising:
inserting a guidewire from the axial lumen through the hole formed by the piercing tip; and
retracting the piercing tip.

30. The method of claim 25 further comprising:
maintaining the threaded dilation portion in the hole when the hole is at a final diameter; and
heating the threaded dilation portion to seal edges of the hole.

31. The method of claim 30 wherein the heating comprises heating by a method selected from the group consisting of inductance heating and resistance heating.

32. A helical screw dilator for graft material of a stent graft comprising:
means for piercing a hole in the graft material;
means for dilating the hole;
means for advancing the dilating means into the hole in response to rotation; and
means for rotating the advancing means.

33. The helical screw dilator of claim 32 further comprising means for receiving a guidewire through the piercing means, the dilating means, and the rotating means.

34. The helical screw dilator of claim 32 further comprising means for retracting the piercing means.
